(19) United States
(12) Plant Patent Application Publication
(10) Pub. No.: US 2006/0218683 P1
(43) Pub. Date: Sep. 28, 2006

(54) HOP PLANT NAMED "01046"

(50) Humulus lupulus L.;

(75) Inventors: Roger D. JESKE, Yakima, WA (US); Joe BRULOTTE, Yakima, WA (US)

Correspondence Address: HAYES, SOLOWAY P.C., 3450 E. SUNRISE DRIVE, SUITE 140, 85718, AZ, 85718, US

(73) Assignee: S.S. Steiner, Inc., New York, NY (US)

(21) Appl. No.: 11/344,245

(22) Filed: Jan. 31, 2006

(51) Int. Cl. A01H 5/00

(52) U.S. Cl. PLT/236

(57) ABSTRACT

Abstract of the Disclosure

A new and distinct variety of hop, Humulus lupulus L., named “BRAVO” is characterized by it’s exceptional cone yield, high percentage of alpha acids, and resistance to hop powdery mildew strains found in Washington. The new variety was cultivated as a result of a cross in 2000 at Golden Gate Roza Hop Ranches in Prosser, Washington, United States and has been asexually reproduced in Prosser, Washington, United States.

### Detailed Description of the Invention

BACKGROUND OF INVENTION

The flowers (cones) of the female hop plant, Humulus lupulus L, are used in the making of beverages, especially beer, as a flavoring and processing component. Hops contribute towards the bitterness and aroma in beer as well as foam quality and flavor and taste stability.

Hop plants, hops cones, male hop flowers, hop plant parts, hop tissue cultures and hop extracts also have bioactive properties; including anti-microbial, anti-cancer, anti-osteoporosis, anti-oxidative, anti-inflammatory, antibiotic, soporific, anti-androgenic, and pro-estrogenic activities, among others; which may be used in herbal remedies, in antimicrobial preparations for food, fodder, food fermentation, food process, animal husbandry; or in non-food uses, such as composting, biofuel processing, fermentation process, water treatment, animal bedding and phytoremediation; and uses in cosmetics, in nutriceutical and in pharmaceutical applications and in research thereof. Examples activities and non-berverage uses are included herein by references: J Hazard Mater. Apr 26;91(1-3):95-112; Mol Cancer Ther. Sep;1(11):959-69; Phytochemistry. May;65(10):1317-30; U.S. Pat. No 6,423,317 (2002); U.S. Pat. No. 6,623,775 (2003).

Agricultural end-product users, growers, handlers and processors of hops, of hop plants, of hop tissues and of hop products use hops and are affected by the agronomic, developmental, morphological, chemical and physical properties that vary among unique and distinct asexually reproduced varieties. This invention relates to an asexually produced hop variety, named BRAVO, invented in a planned and systematically executed breeding program.

SUMMARY OF INVENTION

This invention relates to a new genetically, chemically, and morphologically distinct variety of hop selected from among the multitude of hop plants resulting from a controlled cross-pollination during the summer of 2000. The cross-pollination was between a proprietary non-patented female hop plant referred to as “Zeus” with a proprietary non-patented male hop plant (98004 X USDA 19058m)m. The parentage of “98004” are female hop plant Nugget (commercially available and non-patented) and a non-patented male progeny of open pollinated Nugget. Therefore “BRAVO” parental heritage is 50% Zeus, 18.75% Nugget, 25% USDA 19058m, and 6.25% unknown.

The cross-pollination was between a proprietary non-patented female hop plant referred to as “Zeus” with a proprietary non-patented male hop plant (98004 X USDA 19058m)m. The parentage of “98004” is female hop plant Nugget (commercially available and non-patented) and a non-patented male progeny of open pollinated Nugget.

The cross-pollination resulting in “BRAVO” was performed in 2000 by Roger Jeske, a self-employed agronomist, contracted by S. S. Steiner Inc. All crosses were done at Golden Gate Roza Hop Ranches, Prosser, Washington. During 2001 seeds collected from the cross-pollination were germinated and screened for powdery mildew resistance at the assignee: S. S. Steiner Inc.’s greenhouse Prosser, Washington. These seedlings were planted in a field nursery and further screened for gender, vigor, cone type, and disease resistance with the best genotypes being advanced to a single hill hop nursery during the spring of 2002. Mr. Jeske discovered the “BRAVO” variety during the summer of 2002 among the numerous hop genotypes growing at the S. S. Steiner, Inc. single hill hop nursery. These hop genotypes growing in the single hill hop nursery were grown in a 3.5’ X 14’ spacing on twine attached to an 18’ trellis system.

Chemical analysis and field observations in 2002 demonstrated unusually high percentage of alpha-acids, lack of powdery mildew in the leaves and cones, and an exceptional cone set. Cones were compact and easy to pick with no shatter. Vigor was excellent and yield potential appeared to be exceptional, although yield data was not taken in 2002.

In 2003, “BRAVO” was asexually propagated at the S. S. Steiner, Inc. greenhouses. Rhizomes from the original single hill plant of “BRAVO” were dug, divided and planted into multiple greenhouse grown containers. Softwood cuttings were periodically taken from these original containers until approximately 4,000 softwood-cutting plants were made. These plants constituted the first asexual reproduction of the “BRAVO” variety and are the second generation. They were grown at two geographical locations with no powdery mildew observed. These two small scale trials consisting of a multi-hill planting (7 plants) located at Golden Gate Roza Hop Ranches, Prosser, Washington and a multi-hill planting (7 plants) located at Golden Gate Emerald Hop Ranches, Sunnyside, Washington. These trials were subjected to standard agronomic, cultural and management practices for the purpose of determining harvest ability, yield, chemical characteristic, and process ability. Also, established in 2003 was a large-scale trial of second-generation plants north of Prosser, Washington. This consisted of planting two acres in a 3.5’ X 14’ configuration (1778 hills), with two softwood cuttings of “BRAVO” planted per hill.

During 2003, 2004, and 2005 second-generation plants in the two small-scale trials and one large-scale trial at Golden Gate Hop Ranches were sampled for chemical analysis and harvested for yield evaluations. Results from the test plots provided additional information supporting the disease resistance, yield potential, and alpha-acids projections made from the original “BRAVO” plant (first generation) selected in 2002. Thus confirmed the exceptionally high yield and high alpha-acids percentages of the new variety. The 2003 harvest of greenhouse potted plant baby “BRAVO” yielded 2200 lbs./acre from the two-acre test plot of second-generation plants grown north of Prosser, Washington. The 2004-2005 commercially harvested two-acre test plot of second-generation plants produced an average of 3200 lbs./acre. Commercially harvested hop cones were dried at Golden Gate Roza Hop Ranch kilns to approximately 10% moisture, then were pressed into 200 lb. bales. Bale samples from the 2003-2005 second generation “BRAVO” hops grown in the Prosser location showed an average alpha-acids level of 17.5% and beta-acids level of 3.5% (American Society of Brewing Chemists spectrophotometric method). The percentage of alpha acids is nearly identical to the commercial variety Zeus (mother of “BRAVO”). Storage stability of second generation “BRAVO” had an average loss of alpha-acids of the harvested hop cones stored at room temperature (22⁰C) for six months of 33%. Zeus, the mother of “BRAVO”, by comparison had an average loss of alpha-acids of 48% in identical storage stability trials.

In 2005, the second asexual reproduction of the “BRAVO” variety took place. The second-generation rootstock from the two acres was dug, divided and planted into a larger acreage test plot near Sunnyside, Washington. These third generation plants were grown by Golden Gate Emerald Hop Ranch, utilizing standard agricultural practices that are common for hop production, except no powdery mildew fungicides application was made. Third generation plants in the thirty-five acres at Golden Gate Emerald Hop Ranch were sampled for chemical analysis and harvested for yield evaluations. Results from this third generation large-scale test plot provided additional information supporting the powdery mildew resistance and high alpha-acids percentages. The 2005 yield from these baby “BRAVO” plants was very good as compared to the baby yield of other commercial varieties.

Based on agronomic and chemical evaluations over a number of growing seasons both secondary and tertiary clones (second and third generations) of “BRAVO” exhibited genetic stability with respect to its novel characteristics of complete powdery mildew resistance, very high yield (>3000 lb./ac), and high alpha-acids percentages. This is in contrast to the mother, Zeus, which is very susceptible to powdery mildew.

All observations, evaluations and testing of the “BRAVO” variety's agronomic, morphological, physical, and chemical properties were carried out by or directed by the inventors. Co-operators were Golden Gate Hop Ranches and S. S. Steiner Inc.

The variety “BRAVO” is very late maturing and is usually not ready to pick until 20th to the 30th of September. The compact and ovoid shape cones of this variety are mid sized and very plentiful resulting in easy mechanical picking and cleaning much like Nugget. The cones detach easily from stems, and the cones do not shatter during commercial harvesting and drying.

In order demonstrate genetic and phenotypic distinctiveness to closely related varieties, “BRAVO” is compared to its mother and to other agronomically important varieties. Zeus is the parent of “BRAVO” and therefore similarities would be expected. The primary differences between the new “BRAVO” variety and Zeus is the complete resistant of “BRAVO” to the powdery mildew strains found in the Yakima valley and improved storage stability of alpha acids as compared to Zeus. Also, “BRAVO” mostly exhibits main bine leaves with three lobes as opposed to Zeus main bine leaves that are mostly five lobed. Similarities include similar cone shape and weight of 220-280 mg. at maturity, mature main bine of green coloration with purple stripes, and both varieties having excellent yields (>3000 lbs./ac). Chemical similarities show that both have alpha-acids levels of 16-18% and a humulene/caryophyllene ratio of 1.7

The detailed botanical description and drawings herein below allow distinction of the variety from related varieties. For illustration, comparisons of select distinguishing traits to further selected commercial varieties are set forth in Table 1.

TABLE 1

| | “BRAVO” | Galena | Nugget | Zeus |
|---|---|---|---|---|
| Alpha acids | 14-17 | 10-13.5 | 11-14 | 12-16.5 |
| % w/w | | | | |
| Beta acids | 3.0-5.0 | 7.0-9.0 | 4.0-6.0 | 4.0-6.0 |
| % w/w | | | | |
| CoH % w/w | 29-34 | 35-40 | 24-30 | 27-35 |
| of alpha acids | | | | |
| Total Oil | 1.6-2.4 | 0.9-1.2 | 1.5-3.0 | 1.0-2.0 |
| ml/100 g | | | | |
| Humulene % | 18-20 | 10-15 | 12-22 | 10-25 |
| Caryophyllene | 10-12 | 3.0-5.0 | 7-10 | 5.0-15 |
| % | | | | |
| Powdery | Resistant | Susceptible | Resistant | Susceptible |
| Mildew | | | | |
| Disease | | | | |
| Cone Yield | 2700-3100 | 1600-2220 | 1700-2200 | 2400-3000 |
| lbs/acre | | | | |

BRIEF DESCRIPTION OF THE DRAWINGS

The accompanying photographs illustrates the cones, leaves and growth habit of the new “BRAVO” variety:

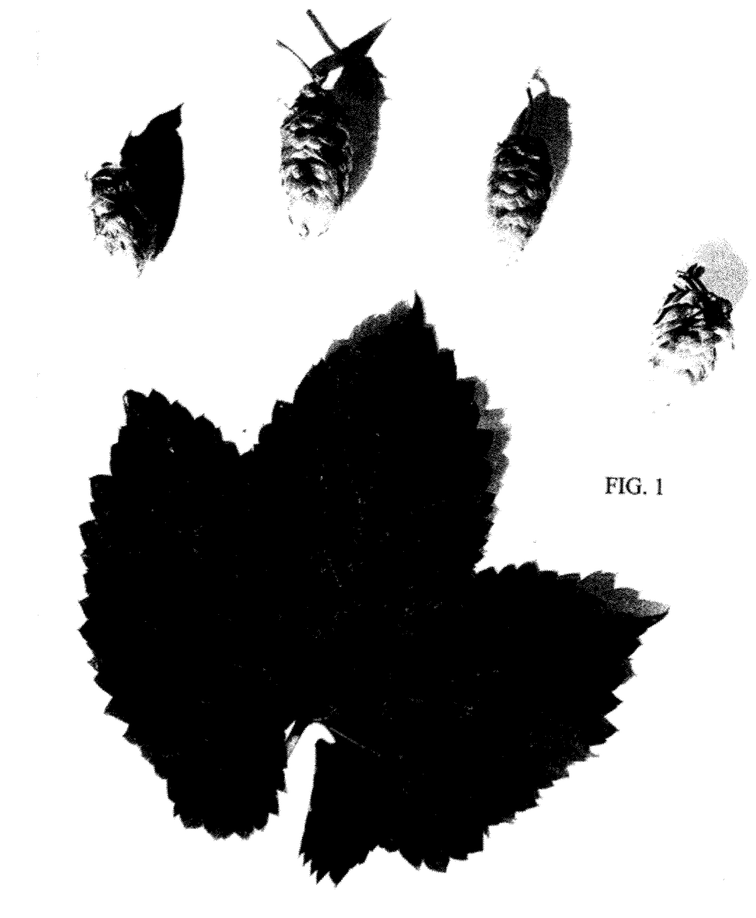

FIG. 1 depicts a close up of whole cones and a mature main vine leaf.

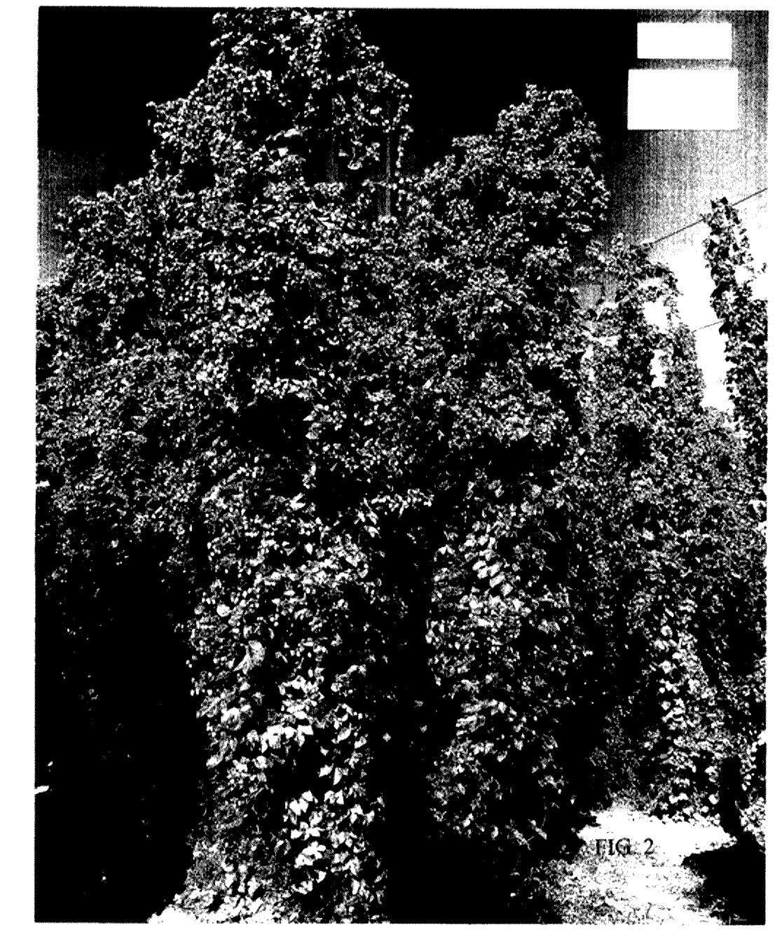

FIG. 2 depicts the appearance of the plants and cones as they are growing in the field on high trellis (18’) approaching harvest time.

GENERAL BOTANICAL DESCRIPTION OF THE VARIETY

This description provides information on agronomic, morphological, chemical and processing characteristics of the new variety that are used in distinction and identification of a new hop variety and its parts and products by practitioners of the industries that use hops as described herein above.

General information on the botanical characteristics and cultural aspects of hops plants as they relate to agronomics, breeding and food use are particularly well discussed in the prior art, especially, United States Patents PP10,956 and PP13,132 and the literature; “Steiner’s Guide to American Hops Book III”, 1986; “Hops” Published by Chapman and Hall, 1991. Detailed botanical information below of the present variety is directly comparable to the prior art, as contained by reference herein.

DETAILED BOTANICAL DESCRIPTION OF THE VARIETY

Following is a detailed description of the botanical and analytical chemical characteristics of the new variety. The information for this botanical description was either collected or verified during the growing seasons of 2002 through 2005 in the growing areas north of Prosser, Washington and south of Sunnyside, Washington. Botanical characteristics, and to a lesser degree the analytical characteristics are somewhat dependent on cultural practices and climatic conditions and can vary with location or year:

1. Parentage: A hop plant originating from a controlled cross-pollination between proprietary non-patented female hop plant referred to as “Zeus” with a proprietary non-patented male hop plant (98004 X USDA 19058m)m.

2. Locality where grown and observed: North of Prosser, Washington and south of Sunnyside, Washington.

3. Agronomic factors: Dates of first and last harvest are approximately September 20 and September 30. Shoots of “BRAVO” emerge from winter dormancy about the same time as the commercial variety Zeus. Emergence is typically by March 15^{th} and the initial stem growth is fairly rapid like Zeus or Nugget. “BRAVO” show good vigor and moderate growth rate. After spring pruning, growth continues to be average when compared to other commercial varieties. Inflorescence of “BRAVO” begins to appear in the middle of July and mature during the forth week of September. Cone shape is fairly uniform in the “BRAVO” variety. The hop cones of “BRAVO” are well adapted to mechanical harvest because of their compactness and ovoid shape. The cones are similar to the variety Nugget and roll nicely on dribble belts, making cleaning easier. The cones do not shatter during harvest.

4. Plant characteristics:

Plant: green vigorous, climbing vine

Plant shape: columnar

Bine stripe: purple

Bine inter-node length (@ 6’ high): 22 cm.

Bine diameter (@ 6’ high): 1 cm.

Petiole length: 6-7 cm.

Petiole shape: slightly channeled (flat upper surface)

Leaf arrangement: opposite

Leaf shape: cordate

Mature leaf width: 14-16 cm.

Number of main bine leaf lobes: three

Leaf margin: moderately serrate to dentate

Lateral length: 0.8-1.0 m.

Lateral inter-node length: 10-13 cm.

5. Cone characteristics:

Bract tips shape: acute to cuspidate

Bract tip position: mostly appressed

Bracteole shape: lanceolate

Compactness: tight to semi-dense

Shape: ovoid

Cone length: 3-4 cm.

Cone tip shape: bluntly pointed

Cone weight: 220-280 mg.

Strig: compact

Yield per acre: 3000-3470 pounds average

Maturity: late to very late

6. Analytical data of cones:

% Alpha-Acids (bale): 14.0-17.0% (ASBC Spectrophotometric method)

% Beta-Acids (bale): 3.0-4.0% (ASBC Spectrophotometric method)

Alpha/beta ratio: 4.5-5.5

Cohumulone (% of alpha-acids): 29-34%

Storage characteristics: 30-35% transformation of alpha acids after 6 months at 22⁰ C

Total oils (mls/100 g): 1.6-2.4

Humulene (% of total oils): 18-20%

Caryophyllene (% of total oils): 10-12%

Humulene/Caryophyllene ratio: 1.7

Farnesene (% of total oils): 0.5%

Myrcene (% of total oils): 25-50%

7. Disease resistance: The variety “BRAVO” is resistant to the strains of powdery mildew fungus found in the Yakima valley as of 2005, but since not all strains are present no future powdery mildew resistance can be assured. “BRAVO” appears to be tolerant to strains of Verticillium wilt and the virus diseases found in the USA grown areas. Susceptible to hop downy mildew fungus is moderate and preventative measures are recommended. Tolerance to the major soil borne pests and diseases that affect hops is not known at this time.

8. Regional adaptation: The “BRAVO” variety is well adapted to the drier growing regions of Washington State, specifically the Yakima Valley. “BRAVO” has not been tested in other growing locations, but hop growing regions like the Willamette Valley of Oregon may not be suitable due to high humidity and the susceptibility of this variety to downy mildew infections.

9. Ploidy: Hop variety “BRAVO” is diploid. The mother is diploid and the father is diploid.

10. Life expectancy: Indefinite similar to other hop varieties.

11. Use: Flavoring and conditioning of beverages and foods and use as vegetable. Constituent of herbal remedies, nutraceuticals, pharmaceuticals, drugs, ointments, antiseptic washes and cosmetics for humans and animals. Constituent of fodder, bedding, compost, agricultural treatments, phytoremediation treatments, water and soil treatments, conditioning of fermentation and other industrial processes. Used in breeding novel hop varieties.

12. Propagation status: “BRAVO” rootstock and plant propagation material exists. Asexual plant propagation has been demonstrated.

13. Reproductive status: “BRAVO” is fertile and produces seeds upon pollination with male hop plants.

DISTINGUISHING CHARACTERISTICS

This new hop variety “BRAVO” can be distinguished from all other USA commercial varieties by its resistance to powdery mildew in combination with a high percentage of alpha-acids and excellent yield.

What is Claimed is:

1. A new variety of hop plant, named BRAVO, uniquely characterized by its resistance to hop powdery mildew strains currently found in Washington hop fields, exceptional yield, and a high percentage of alpha acids.
